(19) United States
(12) Patent Application Publication
(10) Pub. No.: US 2022/0362312 A1
(43) Pub. Date: Nov. 17, 2022

(54) CANNABIDIOL-ENHANCED PROBIOTIC COMPOSITIONS AND USES THEREOF FOR TREATMENT OF INFECTIONS

(71) Applicant: BUZZELET DEVELOPMENT AND TECHNOLOGIES LTD., OR-AKIVA (IL)

(72) Inventors: AHARON EYAL, (US); NOA RAZ, (US)

(21) Appl. No.: 17/754,603

(22) PCT Filed: Oct. 7, 2020
(86) PCT No.: PCT/IB2020/059436

§ 371 Date: Apr. 7, 2022

Related U.S. Application Data
(60) Provisional application No. 62/913,538, filed on Oct. 10, 2019.

(51) Int. Cl. A61K 35/747; A61K 36/185; A61K 31/05; A61K 9/00; A61L 15/36; A61P 1/00; A61P 29/00

(52) U.S. Cl. CPC A61K 35/747; A61K 36/185; A61K 31/05; A61K 9/0034; A61L 15/36; A61P 1/00; A61P 29/00; A61K 2035/115

(57) ABSTRACT

Provided is a pharmaceutical composition for treating a microbial imbalance, the composition comprising a pharmaceutically effective amount of at least one strain of probiotic bacteria, a pharmaceutically effective amount of cannabidiol and a pharmaceutically acceptable excipient. Further provided are feminine hygiene products comprising the composition and methods of use of the composition.

### CROSS-REFERENCE TO RELATED APPLICATIONS

The present application gains priority from U.S Provisional Patent Application Ser. No. 62/913,538 filed 10 Oct. 2019, and U.S Provisional Patent Application Ser. No. 62/937,821 filed 20 Nov. 2019, both of which are incorporated by reference as if fully set-forth herein.

### FIELD OF THE INVENTION

The field of art to which this invention generally pertains is pharmaceutical compositions, and more specifically to pharmaceutical compositions comprising probiotic bacteria and cannabidiol, and uses thereof for treating microbial imbalance and symptoms thereof.

### BACKGROUND OF THE INVENTION

Microbial colonies present in a normal, healthy individual are benign or beneficial, playing a role in digestion and in protecting the body from invasion by pathogenic microbes. Microbial imbalance (also known as dysbiosis or dysbacteriosis) occurs when a part of the human microbiota is altered e.g. by diet, stress or antibiotics. This can manifest in higher occurrence of certain microorganisms, lack of occurrence of certain microorganisms and lack of diversity of microorganisms. An imbalance may occur in any of the microbial sites of the body—including the skin, gut, vagina and nose. Microbial imbalance is associated with a wide range of health problems.

### SUMMARY OF THE INVENTION

According to an aspect of some embodiments of the present invention, there is provided a pharmaceutical composition for use in treating a microbial imbalance in a part of a body of a subject in need thereof, the composition comprising a pharmaceutically effective amount of at least one strain of probiotic bacteria, a pharmaceutically effective amount of cannabidiol and optionally a pharmaceutically acceptable excipient.

According to some embodiments, the amount of cannabidiol is selected to provide a concentration of between 1 and 50 milligram cannabidiol per liter to said part of the body of the subject.

According to some embodiments, the amount of cannabidiol is selected to provide a cannabidiol concentration of less than 0.2 of candida microbial inhibitory concentration (MIC) to said part of the body of the subject.

According to some embodiments, the amount of cannabidiol is selected to provide a cannabidiol concentration of less than 0.2 of Lactobacillus microbial inhibitory concentration (MIC) to said part of the body of the subject.

According to some embodiments, a weight/weight ratio between said probiotic bacteria and said cannabidiol is greater than 10.

According to some embodiments, said cannabidiol comprises crystalline cannabidiol.

According to some embodiments, said probiotic bacteria comprise a Lactobacillus.

According to some embodiments, said probiotic bacteria are selected from the group consisting of Lactobacillus rhamnosus, Lactobacillus fermentum, Lactobacillus crispatus, Lactobacillus iners, Lactobacillus gasseri, Lactobacillus jensenii, Lactobacillus acidophilus, Lactobacillus plantarum, Lactobacillus vaginalis, Lactobacillus casei, Lactobacillus reuteri and combinations thereof.

According to some embodiments, the pharmaceutical composition comprises 10\hat{ }5 to 10\hat{ }13 CFU of said probiotic bacteria per gram.

According to some embodiments, the composition is in the form of a preparation selected from the group consisting of suspensions, sprays, gels, creams, powders, capsules, solutions for lavages, ovules, vaginal inserts, tablets, microencapsulated products, enteric coated products and combinations thereof.

According to some embodiments, the composition is in the form of a preparation selected from the group consisting of capsules, vaginal inserts and tablets and wherein said preparation comprises between 50 and 1000 milligrams of dry probiotic cells.

According to some embodiments, the composition is in the form of a preparation selected from the group consisting of capsules, vaginal inserts and tablets and wherein said preparation comprises between 0.01 and 15 milligrams cannabidiol.

According to some embodiments, said excipient is selected from the group consisting of mucous adhesive excipients, hydrocolloids, thiosulfates, prebiotics, terpenes, herbal preparations, cryoprotectants, preservatives, lubricants, flow agents, binders, diluents, hyaluronic acid and combinations thereof.

According to some embodiments, said excipient comprises at least one terpene selected from the group consisting of pinene, limonene, caryophyllene, caryophyllene oxide, nerolidol, eucalyptol, terpinene, terpineol, myrcene, cycloartenol, citronellol, amyrin, camphene, cymene, Ocimene, humulene, farnesene, guaiol, eudesmol, friedelin, linalool, camphor, menthol, thymol, carvacrol, eugenol, farnesol, citronellol, geraniol, citral, ioneone and combinations thereof.

According to some embodiments, said excipient comprises at least one herbal preparation selected from the group consisting of herbal preparation of anemarrhenae asphodeloides, cimicifuga (actaea) racemosa, coptis chinensis, echinacea spp, hydrastis canadensis, angellica sinesis, larrea mexicana, viburnum prunifolium, vitex agnus castus, trifolium althea officinalis, andrographis paniculata, apium graveolens, arctostaphylos uva-ursi, barosma betulina, coptis chinensis, plantago spp, uncaria tomentosa, zea mays, berberis vulgaris, tymus volgaris, phellodendron spp, tebebuia impitigenisa, thuia accidentalis, Humulus lupulus, Calendula officinalis, Glycyrrhiza glabra and combinations thereof.

According to some embodiments, the part of the body of the subject is selected from the group consisting of the GI tract, the vagina, the oral cavity, the skin, a nail of the subject and combinations thereof.

According to some embodiments, the pharmaceutical composition is provided in a form configured for vaginal administration.

According to some embodiments, the pharmaceutical composition is provided in a form selected from the group consisting of solutions, creams, pastes, masks or body washes.

According to an aspect of some embodiments of the present invention, there is provided an absorbent feminine hygiene product, comprising the pharmaceutical composition as disclosed herein.

According to some embodiments, the absorbent feminine hygiene product is selected from the group consisting of a diaper, a sanitary napkin, a tampon, a panty shield and an incontinence pad.

According to an aspect of some embodiments of the present invention, there is provided a method for treating or preventing a condition caused by a microbial imbalance in a mammal comprising administration of an effective amount of the pharmaceutical composition as disclosed herein.

According to some embodiments, the method comprises administering a first pharmaceutical composition as disclosed herein at a first time and administering a second pharmaceutical composition as disclosed herein at a second time.

According to some embodiments of the method, a weight/weight ratio of said probiotic bacteria to said cannabidiol in said second pharmaceutical composition is smaller than a weight/weight ratio of said probiotic bacteria to said cannabidiol in said first composition.

According to some embodiments of the method, said microbial imbalance causes a vaginal disease and/or a vaginal infection.

According to some embodiments, said vaginal disease is selected from the group consisting of vulvovaginal candidiasis, trichomonas vaginitis, senile vaginitis, non-specific vaginal infection or mixed vaginal infection.

According to some embodiments, the method comprises treating or preventing an infection of the vagina by pathogenic microorganisms, wherein said pathogenic microorganisms are selected from the group consisting of Gardnerella vaginalis, Candida albicans, Candida glabrata and Candida tropicalis, Atopobium vaginae, Staphylococcus aureus, Escherichia coli, Pseudomonas aeruginosa or salmonella.

According to some embodiments, said treating comprises regulating vaginal flora balance.

According to some embodiments, said microbial imbalance comprises at least one selected from the group consisting of fungal infection, candida infection, candidiasis, recurrent candidiasis, vaginoses, vulvovaginal candidiases or recurrent vulvovaginal candidiasis.

According to some embodiments, said microbial imbalance comprises candida infection of the GI track of the subject.

According to some embodiments, said microbial imbalance comprises candida infection of the oral cavity of the subject.

According to an aspect of some embodiments of the present invention, there is provided a method of allowing healthy vaginal flora to be restored in a subject in need thereof, comprising administration of an effective amount of the pharmaceutical composition as disclosed herein.

### BRIEF DESCRIPTION OF THE FIGURES

Some embodiments of the invention are described herein with reference to the accompanying figures. The description, together with the figures, makes apparent to a person having ordinary skill in the art how some embodiments of the invention may be practiced.

In the Figures:

FIG. 1 is a photograph of the antimicrobial effects of 0.5 μg (10 μl solution) CBD and THC, and 2 μg (40 μl of solution) CBD and THC on Escherichia coli (1A); Bacillus Thuringiensis (1B); Staphylococcus Aureus (1C); and Lactobacillus casei (1D).

### DETAILED DESCRIPTION OF THE INVENTION

The present invention relates to pharmaceutical compositions comprising probiotic bacteria and cannabidiol and uses thereof for treating microbial imbalance and symptoms thereof.

As used herein, the term “treating” includes preventing, curing, ameliorating, mitigating, and reducing the instances or severity of a condition or a symptom thereof.

As used herein, the term “microbial imbalance” (also known as dysbiosis) refers to an imbalance in the microbial flora of a subject as compared to that of a healthy individual.

As used herein, the term “probiotic” refers to live microorganisms having beneficial health effects when administered to a subject in adequate amounts.

As used herein, the term ‘CBD’ refers to CBDa (cannabidiolic acid) and/or to CBD (cannabidiol) unless indicated otherwise.

Unless indicated otherwise, the term ‘cannabinoid” as used herein refers to a compound that affects the endocannabinoid system and includes the acid form and the decarboxylated form of the compound, or combinations thereof. Cannabinoids are agonists or antagonists to receptors in the endocannabinoid system.

Unless indicated otherwise, percent is weight percent and ratio is weight/weight ratio. Unless indicated otherwise, weight ratio means the ratio between weight content.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

The use of probiotic compositions is known for balancing microbial imbalance. However, such compositions have shown only limited results. The present inventors have surprisingly found that a composition comprising a probiotic and cannabidiol is highly effective in treating such microbial imbalance. Without wishing to be limited to any one theory, it is hypothesized by the present inventors that the surprising effect is provided due to the combined activity of two different mechanisms, wherein the probiotic competes for resources with undesired microbial flora present in the subject thereby limiting the growth of such flora; while the cannabidiol inhibits formation of a contaminant biofilm at concentrations which are significantly lower than those required for an antimicrobial effect when administered in the absence of a probiotic. Furthermore, it has surprisingly been found that while cannabidiol can be shown to exert an antimicrobial effect against certain genera of bacteria, no antimicrobial effect occurs against the probiotic microorganisms as disclosed herein.

According to an aspect of some embodiments of the present invention, there is provided a pharmaceutical composition comprising a pharmaceutically effective amount of at least one strain of probiotic bacteria, a pharmaceutically effective amount of cannabidiol and optionally a pharmaceutically acceptable excipient for use in treating a microbial imbalance in a part of a body of a subject in need thereof. According to some embodiments, at least a fraction of the amount of said at least one strain of probiotic bacteria is dried. According to some embodiments, at least a fraction of the amount of said at least one strain of probiotic bacteria is freeze-dried.

According to some embodiments, the amount of cannabidiol is selected to provide a concentration of between 1 and 50 milligram cannabidiol per liter to said part of the body of the subject. According to some such embodiments, the amount of cannabidiol is selected to provide about 2 milligrams, about 3 milligrams, about 4 milligrams, about 5 milligrams, about 10 milligrams, about 15 milligrams, about 20 milligrams, about 25 milligrams, about 30 milligrams, about 35 milligrams, about 40 milligrams or about 45 milligrams cannabidiol per liter to said part of the body of the subject.

According to some embodiments, the amount of cannabidiol is selected to provide a cannabidiol concentration of less than 0.2 of candida microbial inhibitory concentration (MIC) to said part of the body of the subject. According to some such embodiments, the amount of cannabidiol is selected to provide a cannabidiol concentration of less than about 0.15, less than about 0.1, less than about 0.05, less than about 0.02 or less than about 0.01 of candida microbial inhibitory concentration (MIC) to said part of the body of the subject

According to some embodiments, the amount of cannabidiol is selected to provide a cannabidiol concentration of less than 0.2 of Lactobacillus microbial inhibitory concentration (MIC) to said part of the body of the subject. According to some such embodiments, the amount of cannabidiol is selected to provide a cannabidiol concentration of less than about 0.15, less than about 0.1, less than about 0.05, less than about 0.02 or less than about 0.01 of Lactobacillus microbial inhibitory concentration (MIC) to said part of the body of the subject

According to some embodiments, a weight/weight ratio between said probiotic bacteria and said cannabidiol is greater than 10. According to some such embodiments, the weight/weight ratio between said probiotic bacteria and said cannabidiol is greater than about 12, greater than about 15, greater than about 20, greater than about 25, greater than about 30, greater than about 35, greater than about 40, greater than about 45 or greater than about 50.

According to some embodiments, said cannabidiol comprises crystalline cannabidiol.

According to some embodiments, said probiotic bacteria comprise a Lactobacillus.

According to some embodiments, said probiotic bacteria are selected from the group consisting of Lactobacillus rhamnosus, Lactobacillus fermentum, Lactobacillus crispatus, Lactobacillus iners, Lactobacillus gasseri, Lactobacillus jensenii, Lactobacillus acidophilus, Lactobacillus plantarum, Lactobacillus vaginalis, Lactobacillus casei, Lactobacillus reuteri and combinations thereof.

According to some embodiments, the pharmaceutical composition comprises 10\hat{ }5 to 10\hat{ }13 CFU of said probiotic bacteria per gram. According to some embodiments, the pharmaceutical composition comprises about 10\hat{ }6, about 10\hat{ }7, about 10\hat{ }68, about 10\hat{ }9, about 10\hat{ }10, about 10\hat{ }11 or about 10\hat{ }12 CFU of said probiotic bacteria per gram.

According to some embodiments, the pharmaceutical composition further comprises at least one additional cannabinoid.

According to some embodiments, said additional cannabinoid is selected from the group consisting of tetrahydrocannabiniol in acid or decarboxylated form (THCa or THC, respectively), cannabidiol in acid or decarboxylated form (CBDa or CBD, respectively), cannabigerol in acid or decarboxylated form (CBGa or CBG, respectively), cannabichromene in acid or decarboxylated form (CBCa or CBC, respectively) tetrahydrocannabivarin in acid or decarboxylated form (THCVa or THCV, respectively), Cannabidivarin in acid or decarboxylated form (CBDVa or CBDV respectively), Cannabinol in acid or decarboxylated form (CBNa or CBN, respectively), Cannabicyclol in acid or decarboxylated form (CBLa or CBL, respectively), Cannabivarin in acid or decarboxylated form (CBVa or CBV, respectively) and combinations thereof.

According to some embodiments, the composition is in the form of a preparation selected from the group consisting of suspensions, sprays, gels, creams, powders, capsules, solutions for lavages, ovules, vaginal inserts, tablets, microencapsulated products, enteric coated products and combinations thereof.

According to some embodiments, the composition is in the form of a preparation selected from the group consisting of capsules, vaginal inserts and tablets and wherein said preparation comprises between 50 and 1000 milligrams of dry probiotic cells. According to some such embodiments, said preparation comprises about 60, about 70, about 80, about 90, about 100, about 200, about 300, about 400, about 500, about 600, about 700, about 800, about 900, about 1000 milligrams of dry probiotic cells.

According to some embodiments, the composition is in the form of a preparation selected from the group consisting of capsules, vaginal inserts and tablets and wherein said preparation comprises between 0.01 and 15 milligrams cannabidiol. According to some such embodiments, the preparation comprises about 0.02, about 0.03, about 0.04, about 0.05, about 0.1, about 0.2, about 0.3, about 0.4, about 0.5, about 1.0, about 2.0, about 5.0 or about 10 milligrams cannabidiol.

According to some embodiments, said excipient is selected from the group consisting of mucous adhesive excipients, hydrocolloids, thiosulfates, prebiotics, terpenes, herbal preparations, cryoprotectants, preservatives, lubricants, flow agents, binders, diluents and combinations thereof.

According to some embodiments, said excipient comprises at least one terpene selected from the group consisting of pinene, limonene, caryophyllene, caryophyllene oxide, nerolidol, eucalyptol, terpinene, terpineol, myrcene, cycloartenol, citronellol, amyrin, camphene, cymene, Ocimene, humulene, farnesene, guaiol, eudesmol, friedelin, linalool, camphor, menthol, thymol, carvacrol, eugenol, farnesol, citronellol, geraniol, citral, ioneone and combinations thereof.

According to some embodiments, said excipient comprises at least one herbal preparation selected from the group consisting of herbal preparation of anemarrhenae asphodeloides, cimicifuga (actaea) racemosa, coptis chinensis, echinacea spp, hydrastis canadensis, angellica sinesis, larrea mexicana, viburnum prunifolium, vitex agnus castus, trifolium althea officinalis, andrographis paniculata, apium graveolens, arctostaphylos uva-ursi, barosma betulina, coptis chinensis, plantago spp, uncaria tomentosa, zea mays, berberis vulgaris, tymus volgaris, phellodendron spp, tebebuia impitigenisa, thuia accidentalis and combinations thereof.

According to some embodiments, the part of the body of the subject is selected from the group consisting of the GI tract, the vagina, the oral cavity, the skin, a nail of the subject, and combinations thereof.

According to some embodiments, the part of the body of the subject is the vagina. In some such embodiments, the microbial imbalance comprise an infection by a microorganism selected from the group consisting of Gardnerella vaginalis, Candida albicans, Candida glabrata and Candida tropicalis, Atopobium vaginae, Staphylococcus aureus, Escherichia coli, Pseudomonas aeruginosa or salmonella. In a preferred embodiment, the microbial imbalance comprises an infection by microorganisms of the genus Candida.

According to some embodiments, the pharmaceutical composition is provided in a form configured for vaginal administration.

According to some embodiments, the pharmaceutical composition is provided in a form selected from the group consisting of solutions, creams, pastes, masks or body washes.

According to some embodiments, the pharmaceutical composition is devoid of a fatty acid amide. According to some embodiments, the pharmaceutical composition is devoid of spilanthol.

According to some embodiments, the pharmaceutical composition is devoid of a polyphenol. According to some embodiments, the pharmaceutical composition is devoid of punicalagin.

According to an aspect of some embodiments of the present invention, there is provided an absorbent feminine hygiene product, comprising the pharmaceutical composition as disclosed herein.

According to some embodiments, the absorbent feminine hygiene product is selected from the group consisting of a diaper, a sanitary napkin, a tampon, a panty shield and an incontinence pad.

According to an aspect of some embodiments of the present invention, there is provided kit comprising said pharmaceutically effective amount of at least one strain of probiotic bacteria, and said pharmaceutically effective amount of cannabidiol.

According to an aspect of some embodiments of the present invention, there is provided a method for treating or preventing a condition caused by a microbial imbalance in a mammal comprising administration of an effective amount of the pharmaceutical composition as disclosed herein.

According to an aspect of some embodiments of the present invention, said mammal is a women at menopausal age.

According to an aspect of some embodiments of the present invention, there is provided a composition as disclosed herein for use in treating or preventing a condition caused by a microbial imbalance in a mammal.

According to some embodiments, the method or use comprises administering a first pharmaceutical composition as disclosed herein at a first time and administering a second pharmaceutical composition as disclosed herein at a second time.

According to some embodiments of the method or use, a weight/weight ratio of said probiotic bacteria to said cannabidiol in said second pharmaceutical composition is smaller than a weight/weight ratio of said probiotic bacteria to said cannabidiol in said first composition.

According to some embodiments of the method or use, said microbial imbalance causes a vaginal disease and/or a vaginal infection.

According to some embodiments of the method or use, said vaginal disease is selected from the group consisting of vulvovaginal candidiasis, trichomonas vaginitis, senile vaginitis, non-specific vaginal infection or mixed vaginal infection.

According to some embodiments, the method or use comprises treating or preventing an infection of the vagina by pathogenic microorganisms, wherein said pathogenic microorganisms are selected from the group consisting of Gardnerella vaginalis, Candida albicans, Candida glabrata and Candida tropicalis, Atopobium vaginae, Staphylococcus aureus, Escherichia coli, Pseudomonas aeruginosa or salmonella.

According to a preferred embodiment of the method or use, the microorganisms are of the genus Candida.

According to some embodiments of the method or use, said treating comprises regulating vaginal flora balance.

According to some embodiments of the method or use, said microbial imbalance comprises at least one selected from the group consisting of fungal infection, bacterial infection, candida infection, candidiasis, recurrent candidiasis, vaginoses, vulvovaginal candidiases or recurrent vulvovaginal candidiasis.

According to some embodiments of the method or use, said microbial imbalance comprises candida infection of the GI track of the subject.

According to some embodiments of the method or use, said microbial imbalance comprises candida infection of the oral cavity of the subject.

According to an aspect of some embodiments of the present invention, there is provided a method of allowing healthy vaginal flora to be restored in a subject in need thereof, comprising administration of an effective amount of the pharmaceutical composition as disclosed herein.

The scope of the invention shall include all modifications and variations that may fall within the scope of the attached claims. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### EXAMPLE 1

### Representative Compositions

TABLE 1

| | | Dried | | | |
|---|---|---|---|---|---|
| Example | | bacteria | Crystalline | | |
| number | Bacteria | content | CBD content | | Terpenes |
| 1 | Lactobacillus | 50 mg | 0.01 | mg | pinene, |
| | rhamnosus | | | | limonene, |
| | | | | | caryophyllene |
| 2 | Lactobacillus | 100 mg | 0.05 | mg | / |
| | vaginalis | | | | |
| 3 | Lactobacillus | 200 mg | 0.1 | mg | linalool, |
| | crispatus + | (total) | | | camphor, |
| | Lactobacillus | | | | menthol, |
| | casei | | | | thymol |
| 4 | Lactobacillus | 300 mg | 0.5 | mg | humulene, |
| | reuteri + | (total) | | | farnesene, |
| | Lactobacillus | | | | guaiol, |
| | gasseri + | | | | eudesmol, |
| | Lactobacillus | | | | friedelin |
| | jensenii | | | | |
| 5 | Lactobacillus | 400 mg | 1 | mg | / |
| | crispatus | | | | |
| 6 | Lactobacillus | 500 mg | 2 | mg | / |
| | iners | | | | |
| 7 | Lactobacillus | 600 mg | 5 | mg | citronellol, |
| | acidophilus, | (total) | | | amyrin, |
| | Lactobacillus | | | | camphene, |
| | plantarum | | | | cymene |
| 8 | Lactobacillus | 700 mg | 6 | mg | citronellol, |
| | reuteri | | | | geraniol, |
| | | | | | citral, |
| | | | | | ioneone |
| 9 | Lactobacillus | 800 mg | 7 | mg | menthol, |
| | casei | | | | thymol, |
| | | | | | carvacrol, |
| | | | | | eugenol, |
| | | | | | farnesol |
| 10 | Lactobacillus | 1000 mg | 8 | mg | camphene, |
| | plantarum | | | | cymene, |
| | | | | | Ocimene, |
| | | | | | humulene |

### EXAMPLE 2

### Effect of Cannabidiol in Treating Candida albicans Infection

A study was performed to evaluate the efficacy of cannabidiol in treating or preventing an infection by pathogenic microorganisms comprising Candida albicans, via preventing biofilm formation.

Media of Candida albicans grown in RPMI medium supplemented with 1% glucose were introduced into wells of a 24-well plate CBD solutions in olive oil were added to each well. CBD concentrations in the added CBD solutions ranged between 30 and 10,000 mg/L. After incubation for 24 hours, spent medium and free-floating fungi were removed by gentle aspiration and the wells were washed twice with phosphate-buffered saline (PBS, pH 7.4).

The presence of biofilm was then quantified by crystal violet staining. Briefly, 0.02% crystal violet dye was added into each of the wells for 45 minutes and the wells were then washed twice with double distilled water (DDW) to remove unbound dye. 200 μl of 30% acetic acid was then added into each well, the plate was shaken for 10 minutes to release the dye and the biofilm was quantified by measuring the absorbance at 595 nm using a Genious plate reader Spectrophotometer (Tecan).

Cannabidiol was shown to have a very strong inhibitory effect on Candida albicans biofilm formation, exhibiting their minimal biofilm inhibitory concentration (MBIC) at between 30 and 80 mg/L.

### EXAMPLE 3

### Antibacterial Effect of CBD and THC on Various Microorganisms

The anti-bacterial effect of CBD and THC was assessed on Escherichia coli, Bacilus Thuringiensis, Staphylococcus Aureus and lactobacillus Casei as follows:

Materials and Methods

CBD source extract: 57% CBD

THC source extract: 41.4% THC.

Both extracts were prepared by ethanol extraction followed by ethanol evaporation. Cannabinoids concentrations were determined by high-performance liquid chromatography (HPLC).

Solutions were prepared using 5% ethanol solutions.

Suspensions of approximately 10⁴-10⁶ cells (100 ul bacterial suspension OD₆₀₀˜0.6-0.8) were prepared for each of Escherichia coli, Bacillus Thuringiensis, Staphylococcus Aureus and lactobacillus Casei cells. For Escherichia coli, Bacillus Thuringiensis and Staphylococcus Aureus, Tryptic Soy Agar (BD) was used as the medium for testing, and Tryptic Soy Broth (BD) for inoculant growth. For Lactobacillus casei, Lactobacillus media (BD) was used for both.

Cells were allowed to grow for 48 hours at 30° C.

The 4 types of bacterial were each contacted with 0.5 μg CBD, 2 μg CBD, 0.5 μg THC and 2 μg THC and the anti-bacterial effect for each microorganism tested was assessed using the standard disc-diffusion agar method such as described in the Kirby-Bauer Disk Diffusion Susceptibility Test Protocol as published by the Clinical Laboratory Standards Institute (Performance Standards for Antimicrobial Disc Susceptibility Tests: Approved Standard, 9^{th} Edition), which is incorporated by reference as if fully set out herein.

The discs were coated with 10 μl solution for each of 0.5 μg CBD and 0.5 μg THC, and with 40 μl of solution for each of 2 μg CBD and 2 μg THC.

The discs were allowed to dry for 10 minutes at ambient temperature and pressure. Following drying the discs were placed upside down on each dish with specific bacterial growth media.

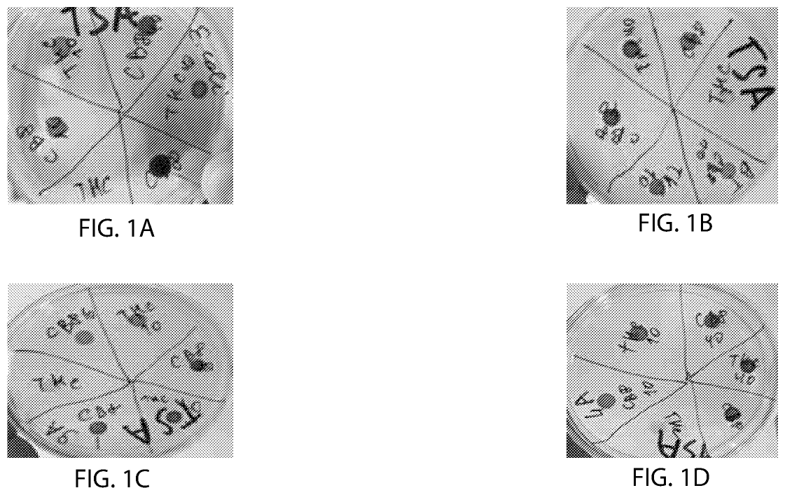

Results are shown in FIG. 1 and in Table 2 below, where ‘+’ indicates growth inhibition and ‘−’ indicates no growth inhibition and wherein a result in millimeters refers to the area of inhibited bacterial growth. As shown, CBD and THC at the concentrations tested has no effect on growth of Escherichia coli, Staphylococcus Aureus or lactobacillus Casei, but do inhibit growth of Bacillus Thuringiensis.

TABLE 2

| | CBD | CBD | THC | THC |
|---|---|---|---|---|
| Bacteria | 0.5 μg | 2 μg | 0.5 μg | 2 μg |
| Escherichia | − | − | − | − |
| coli | | | | |
| Bacillus | +2 mm | +3 mm | +1 mm | +1 mm< |
| Thuringiensis | | | | |
| Staphylococcus | − | − | − | − |
| Aureus | | | | |
| Lactobacillus | − | − | − | − |
| casei | | | | |

### EXAMPLE 4

### Effect of a Composition Comprising Cannabidiol and Lactobacillus casei in Treating Candida albicans Infection

A study is performed to evaluate the efficacy of a composition comprising cannabidiol and lactobacillus Casei in treating or preventing an infection by pathogenic microorganisms comprising Candida albicans, via preventing biofilm formation.

RPMI medium supplemented with 1% glucose is introduced into each well of a 24-well plate.

Candida albicans contacted with cannabidiol are added to each well of row 1.

Candida albicans contacted with lactobacillus Casei are added to each well of row 2.

Candida albicans contacted with cannabidiol and lactobacillus Casei are added to each well of row 3.

Candida albicans alone are added to each well of row 4.

After incubation for 24 hours, spent medium and free-floating fungi are removed by gentle aspiration and the wells washed twice with phosphate-buffered saline (PBS, pH 7.4).

The presence of biofilm is quantified by crystal violet staining as described above for Example 3.

Biofilm formation is found to be significantly inhibited in each well of rows 1 and 2 as compared to those of row 4, while a significantly greater inhibition is found in each well of row 3 as compared to those of either row 1 or row 2, indicating a combined effect of a composition comprising both a cannabinoid and probiotic bacteria. This result is considered surprising in view of the fact that the probiotic is not inhibited by the antibiotic activity of cannabinoids.

Discussion

As seen in Examples 2 and 3 above, it has surprisingly been found that CBD and THC, which have strong inhibitory effects on growth of various bacteria, did not inhibit growth of probiotic organisms in the composition of the present invention.

The effect of a composition comprising a combination of a cannabinoid and probiotic bacteria on the treatment of Candida albicans is greater than that obtained with either of the components alone.

1. A pharmaceutical composition comprising a pharmaceutically effective amount of at least one strain of probiotic bacteria, a pharmaceutically effective amount of cannabidiol, optionally comprising crystalline cannabidiol, and optionally a pharmaceutically acceptable excipient.

2. The pharmaceutical composition of claim 1, wherein at least a fraction of the amount of said at least one strain of probiotic bacteria is freeze-dried.

3. The pharmaceutical composition of claim 1, wherein the amount of cannabidiol is selected to provide a concentration of between 1 and 50 milligram cannabidiol per liter to said part of the body of the subject.

4. The pharmaceutical composition of claim 1, wherein the amount of cannabidiol is selected to provide a cannabidiol concentration of less than 0.2 of candida albicans microbial inhibitory concentration (MIC) and/or less than 0.2 of Lactobacillus microbial inhibitory concentration (MIC) to said part of the body of the subject.

5. (canceled)

6. The pharmaceutical composition of claim 1, wherein a weight/weight ratio between said probiotic bacteria and said cannabidiol is greater than 10.

7. (canceled)

8. The pharmaceutical composition of claim 1, wherein said probiotic bacteria comprise a Lactobacillus, optionally selected from the group consisting of Lactobacillus rhamnosus, Lactobacillus fermentum, Lactobacillus crispatus, Lactobacillus iners, Lactobacillus gasseri, Lactobacillus jensenii, Lactobacillus acidophilus, Lactobacillus plantarum, Lactobacillus vaginalis, Lactobacillus casei, Lactobacillus reuteri and combinations thereof.

9. (canceled)

10. The pharmaceutical composition of claim 1, comprising 10\hat{ }5 to 10\hat{ }13 CFU of said probiotic bacteria per gram.

11. The pharmaceutical composition of claim 1, wherein said composition is in the form of a preparation selected from the group consisting of suspensions, sprays, gels, creams, powders, capsules, solutions for lavages, ovules, vaginal inserts, tablets, microencapsulated products, enteric coated products and combinations thereof, optionally wherein said composition is in the form of a preparation selected from the group consisting of capsules, vaginal inserts and tablets and said preparation comprises between 50 and 1000 milligrams of dry probiotic cells and/or said preparation comprises between 0.01 and 15 milligrams cannabidiol.

12. (canceled)

13. (canceled)

14. (canceled)

15. The pharmaceutical composition of claim 1, wherein said excipient comprises at least one terpene selected from the group consisting of pinene, limonene, caryophyllene, caryophyllene oxide, nerolidol, eucalyptol, terpinene, terpineol, myrcene, cycloartenol, citronellol, amyrin, camphene, cymene, Ocimene, humulene, farnesene, guaiol, eudesmol, friedelin, linalool, camphor, menthol, thymol, carvacrol, eugenol, farnesol, citronellol, geraniol, citral, ioneone and combination thereof and/or at least one herbal preparation selected from the group consisting of anemarrhenae asphodeloides, cimicifuga (actaea) racemosa, coptis chinensis, echinacea spp, hydrastis canadensis, angellica sinesis, larrea mexicana, viburnum prunifolium, vitex agnus castus, trifolium althea officinalis, andrographis paniculata, apium graveolens, arctostaphylos uva-ursi, barosma betulina, coptis chinensis, plantago spp, uncaria tomentosa, zea mays, berberis vulgaris, tymus volgaris, phellodendron spp, tebebuia impitigenisa, thuia accidentalis and combinations thereof.

16. (canceled)

17. (canceled)

18. The pharmaceutical composition of claim 1, provided in a form configured for vaginal administration, optionally selected from the group consisting of solutions, creams, pastes, masks or body washes.

19. (canceled)

20. An absorbent feminine hygiene product, comprising the pharmaceutical composition of claim 1, optionally selected from the group consisting of a diaper, a sanitary napkin, a tampon, a panty shield and an incontinence pad.

21. (canceled)

22. A kit comprising a pharmaceutically effective amount of at least one strain of probiotic bacteria, a pharmaceutically effective amount of cannabidiol according to claim 1.

23. A method for treating or preventing a condition caused by a microbial imbalance in a mammal comprising administration of an effective amount of the pharmaceutical composition of claim 1, optionally wherein said mammal is a woman at menopausal age.

24. (canceled)

25. The method of claim 23, comprising administering a first pharmaceutical composition according to claim 1 at a first time and administering a second pharmaceutical composition according to Claim at a second time, optionally wherein a weight/weight ratio of said probiotic bacteria to said cannabidiol in said second pharmaceutical composition is smaller than a weight/weight ratio of said probiotic bacteria to said cannabidiol in said first composition.

26. (canceled)

27. The method of claim 23, wherein said microbial imbalance causes a vaginal disease and/or a vaginal infection, optionally selected from the group consisting of vulvovaginal candidiasis, trichomonas vaginitis, senile vaginitis, non-specific vaginal infection or mixed vaginal infection.

28. (canceled)

29. The method of claim 23, comprising treating or preventing an infection of the vagina by pathogenic microorganisms, wherein said pathogenic microorganisms are selected from the group consisting of Gardnerella vaginalis, Candida albicans, Candida glabrata and Candida tropicalis, Atopobium vaginae, Staphylococcus aureus, Escherichia coli, Pseudomonas aeruginosa or salmonella.

30. (canceled)

31. The method of claim 23, wherein said microbial imbalance comprises at least one selected from the group consisting of fungal infection, bacterial infection, candida infection, candidiasis, recurrent candidiasis, vaginoses, vulvovaginal candidiases or recurrent vulvovaginal candidiasis.

32. The method of claim 23, wherein said microbial imbalance comprises candida infection of the GI track and/or of the oral cavity of the subject.

33. (canceled)

34. A method of allowing healthy vaginal flora to be restored in a subject in need thereof, comprising administration of an effective amount of said pharmaceutical composition of claim 1.
